(19) United States
(12) Patent Application Publication
(10) Pub. No.: US 2009/0287165 A1
(43) Pub. Date: Nov. 19, 2009

(54) Cannulated Device with Atraumatic Tip for Delivering Local Depot to a Joint Space and Methods of Use Thereof

(75) Inventors: Susan Drapeau, Memphis, TN (US); Nelson Scarborough, Andover, MA (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/120,819

(22) Filed: May 15, 2008

(51) Int. Cl. A61M 25/00

(52) U.S. Cl. 604/264

(57) ABSTRACT

Devices for delivery of a pharmaceutical depot is provided. Exemplary device may comprise a cannula defining a working channel between a distal end and a proximal end, wherein the distal end comprises an atraumatic tip. The atraumatic tip may comprise a rounded tip or a soft tip. The cannulated delivery device may include a docking member proximally adjacent to the distal end of the lumen. Methods of using such devices to deliver pharmaceutical depot into a joint space are also provided.

### FIELD OF INVENTION

This invention relates to cannulated devices for delivery of a pharmaceutical depot to a surgery site and methods of using such devices.

### BACKGROUND

Traditional surgical procedures for pathologies located deep within the body can cause significant trauma to the intervening tissues. These open procedures often require a long incision, extensive muscle stripping, prolonged retraction of tissues, denervation and devascularization of tissue. Most of these surgeries require several hours of recovery room time and several weeks of post-operative recovery time due to the use of general anesthesia and the destruction of tissue during the surgical procedure. In some cases, these invasive procedures lead to permanent scarring and pain that can be more severe than the pain leading to the surgical intervention.

To combat these problems, minimally invasive surgical techniques have been developed. These techniques, including arthroscopic techniques, reduce pain, post-operative recovery time and the destruction of healthy tissue. Orthopedic patients have particularly benefited from minimally invasive surgical techniques. The site of pathology is accessed through portals rather than through a significant incision, thus preserving the integrity of the intervening tissues. These minimally invasive techniques also often require only local anesthesia. The avoidance of general anesthesia reduces post-operative recovery time and the risk of complications.

Nonetheless, there is still a need in the art for further improvements in tools and methods for minimally invasive surgery.

### SUMMARY

The present invention fills the foregoing need by providing improved tools and methods for minimally invasive surgical procedures.

In one aspect, devices for delivery of pharmaceutical depots are provided. Such devices may comprise a cannula defining a working channel between a distal end and a proximal end, wherein the distal end comprises an atraumatic tip. The atraumatic tip may comprise a rounded tip or a soft tip. The cannulated delivery device may include a docking member proximal to the distal end of the cannula.

The outside diameter of the cannula may decrease toward the distal end. In some embodiments, the cannula may comprise a series of removable nesting tubes slidable relative to each other.

In another aspect, a method for delivering therapy into a joint space is provided. Such method may comprise dilating the tissue of a capsule wall of the joint space with a cannulated delivery device, as described above, and delivering a depot into the joint space. The depot may be anchored in place inside the joint space. In addition, if necessary, the dilated opening in the capsule wall may be closed with a depot or a plug. The plug may be inserted into the joint space through the cannulated device, pulled through the opening in the joint capsule wall while removing the cannulated device from the joint space, and optionally sutured to the capsule wall.

In one embodiment, the capsule wall may be dilated with a cannulated delivery device comprising removable nesting tubes slidable relative to each other. Alternatively, individuals tubes may be employed. Progressively larger tubes may be slid through the capsule wall until the wall is open to t desired size, at which time one or all inner tubes may be removed to define a working channel.

In yet another aspect, a method for delivering therapy into a joint space may comprise docking a first cannulated device to an outer wall of the joint capsule, making a cut in the wall, and introducing a depot into the joint space. The depot may be introduced either through the first cannulated delivery device or through the second cannulated delivery device advanced into the joint space through the first cannulated delivery device and the hole in the capsule wall.

### BRIEF DESCRIPTION OF THE FIGURES

FIGS. 1a, 1b, and 1c illustrate various embodiments of cannulated delivery devices disclosed herein.

FIG. 2 depicts an embodiment of the cannulated delivery device with a rounded tip.

FIGS. 3a and 3b depict embodiments of the cannulated delivery device with a soft tip.

FIGS. 4a and 4b depict an embodiment of the cannulated delivery device with a retractable soft tip.

FIGS. 5a, 5b and 5c depict various embodiment of docking members.

FIGS. 6a through 6d depict one possible method of delivering a pharmaceutical depot to a joint space.

### DETAILED DESCRIPTION

In one aspect of the invention, cannulated delivery devices are provided. Such devices may be employed to deliver local pharmaceutical depots to surgical sites. In preferred embodiments, the instant devices are employed to deliver depots into a joint space, inside the joint capsule. By way of general overview, the joint capsule is an envelope surrounding a synovial joint such as knee, spine, shoulder, elbow, fingers, wrist, and so forth. Herein, the space surrounded by the joint capsule will be referred to as joint space. Each capsule consists of an outer layer (stratum fibrosum) composed of white fibrous tissue and an inner layer (stratum synoviale) which is a secreting layer. The inner layer is usually described separately as the synovial membrane.

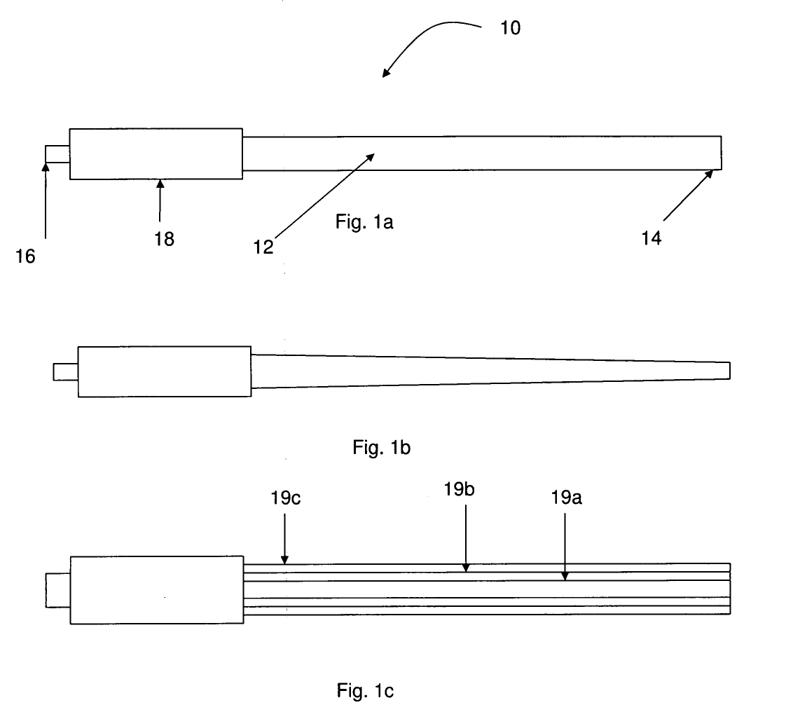

Referring to FIG. 1a, cannulated delivery device 10 generally comprises a cannula 12 defining a working channel between an atraumatic distal end 14 and a proximal end 16 of the cannulated delivery device. The cross-section of the cannula may be selected from, for example, a vertical or horizontal oval, circle, rhombus, and so forth. The length of the cannula 12 is sized so the proximal end of the cannulated device is positioned above the skin of the patient while the distal end of the cannulated device is positioned at the surgical site. The cannulated delivery device may also include a handle 18 that allows the practitioner to control the cannulated delivery device.

In some embodiments, the cannula 12 may have a uniform outside diameter, as shown in FIG. 1a. In other embodiments, the outside diameter of the cannula 12 may taper toward the atraumatic distal end 14, as shown in FIG. 1b. In yet other embodiments, the cannula 12 may comprise a series of nesting tubes 19a, 19b, and 19c that are slidable relative to each other, as shown in FIG. 1c. In preferred embodiments, all nesting tubes have atraumatic distal ends. Each tube may have a uniform or tapering outside diameter independently of the other tubes. In addition, the length of the tubes may be the same or different.

The number of nesting tubes, their diameter and the difference in the diameter between neighboring nesting tubes may vary depending on many factors, including, but not limited to, the type of the joint space to be accessed with the device, the size and condition of the patient, the size of the desired opening, surgeon preferences, and so forth. Accordingly, the nesting tubes may be added or removed to the cannulated device as necessary before or during the procedure. In particular, one or more inner nesting tubes may be capable of being removed from the larger nesting tubes to define one or more working channels into the joint space.

The working channel is sized to receive a medical device and is, preferably, configured to provide a maximum amount of space for delivering medical devices to the surgical site. Example devices inserted through the working channel may include, but are not limited, to pharmaceutical depots, tissue dilators, tissue cutting instruments, guide wires, irrigation devices, cameras, hemostasis devices, plugs, and so forth. In some embodiments, the working channel may be configured to simultaneously receive a plurality of devices. Although the plurality of the devices may be inserted through the same lumen, in some embodiments, the working channel may comprise multiple lumens of the same or different inner diameter or shape. For example, in embodiment depicted in FIG. 1c, it can be achieved by leaving in place several nesting tubes. In other embodiment, cannulas with multiple lumens may be utilized.

The cannulated delivery device may be made of plastic or metal. In some embodiments, the outer surface of the cannulated delivery device may be coated with a lubricating coating to allow the device to more easily move in the patient's body. Such coatings are well known in the art and disclosed in U.S. Pat. Nos. 6,287,285 and 7,264,859, among many others. For example, outer surface of the cannulated delivery device may be coated with a Teflon based coating.

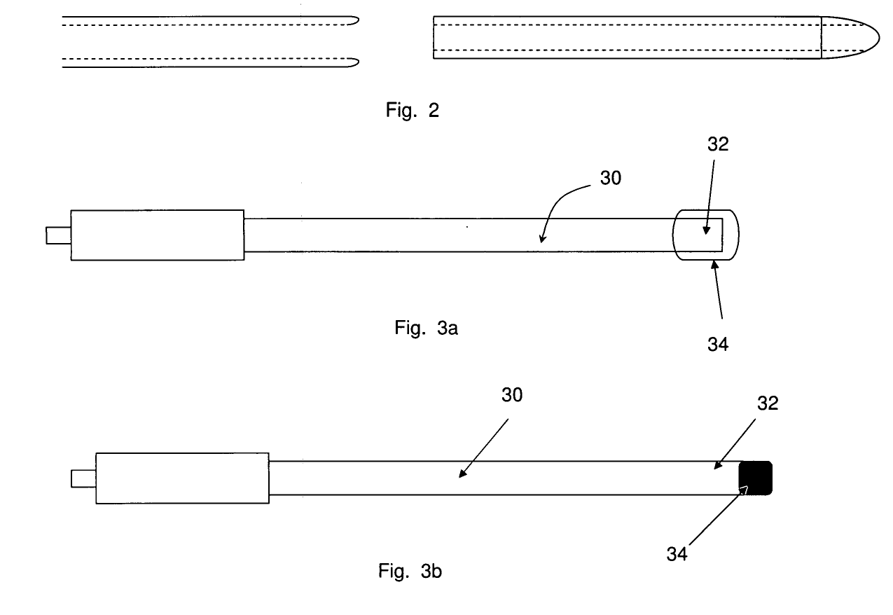

The distal tip of the instant delivery device is atraumatic to the soft tissue outside the joint capsule as well as tissue inside the joint capsule such as articular cartilage. In some embodiments, the distal end of the cannulated delivery device may have a rounded tip which may be achieved by machining or molding the distal tip to eliminate sharp edges. FIG. 2 presents two suitable non-limiting examples for rounded distal tips. In other embodiments, as shown in FIG. 3, the distal tip 32 of the cannulated device 30 may comprise a soft tip 34 that covers the metal tip of the cannula. The soft tip may be made of flexible or compressible material, such as, for example, soft plastic or rubber. Preferably, the outer diameter of the soft tip is the same or substantially the same as the outer diameter of the distal tip of the cannula.

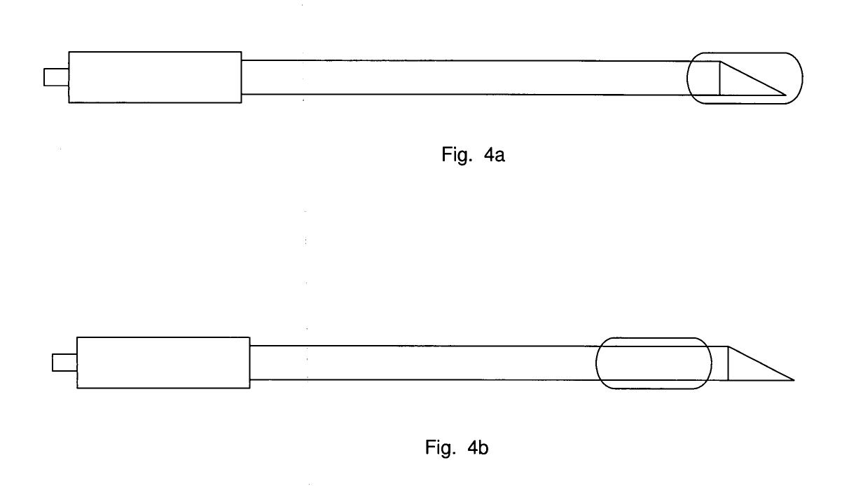

In some embodiments, the soft tip may be stationary. The inner diameter of the soft tip may be dimensioned to fit snuggly over the outer diameter of the distal tip of the cannula in order to prevent it from accidentally slipping during the procedure. Alternatively or additionally, the soft tip may be fixed in place mechanically or using adhesives. In other embodiments, the soft tip may be retracted to expose the distal end of the cannula. In such embodiments, the distal tip of the cannulated delivery device may have a sharp tip that can be used to penetrate tissue, such as a wall of a joint capsule. FIG. 4a shows a cannulated delivery device 40 with a sharp distal end 42 covered by a soft tip 44. As shown in FIG. 4b, the soft tip may be retracted to expose the sharp end of the cannulated delivery device. The sharp end of the cannulated delivery device may be used to penetrate a capsule wall. The sharp edge can be covered with the soft tip before the cannulated device is advanced into the joint space.

It may be desirable to pull the capsule away from the underlying joint tissue while penetrating the capsule. This may prevent damage to the underlying soft tissues such as the cartilage surface. In addition, this technique also allows for a clean, localized cut through the capsule wall which is easier to close and may heal faster. Accordingly, the cannulated delivery device may also include one or more docking members disposed near or at the distal tip to attach the cannulated delivery device to the capsule wall, and to keep the device secure against the capsule wall while the wall is being penetrated. In various embodiments, the docking member may include, but is not limited to, one or more retractable hooks or needles, permanent or temporary adhesive, or one or more suction devices. Once the cannulated device is attached to the capsule, it can be used to pull the capsule away from the underlying joint tissue.

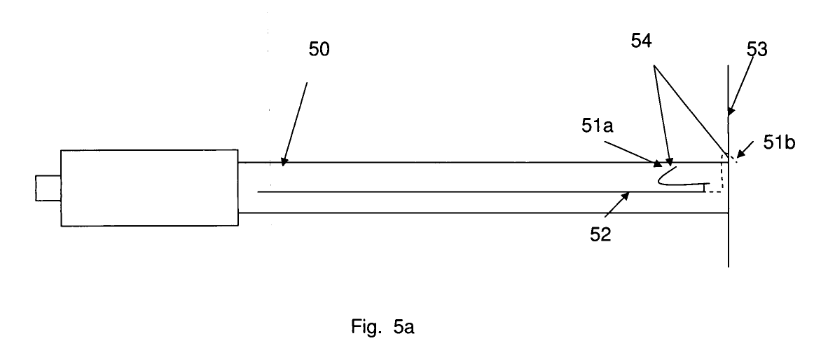

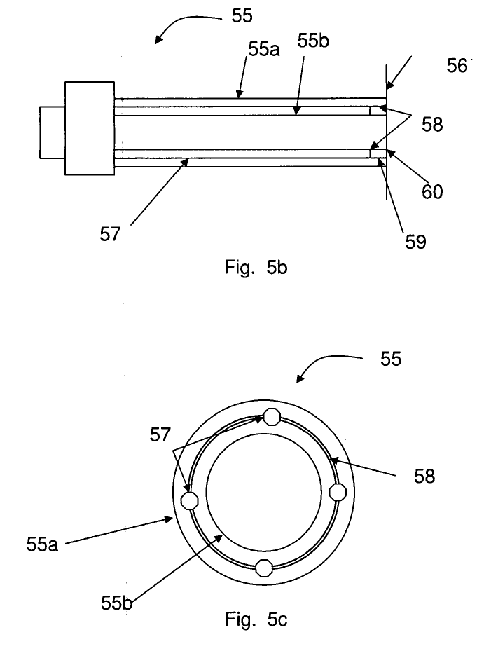

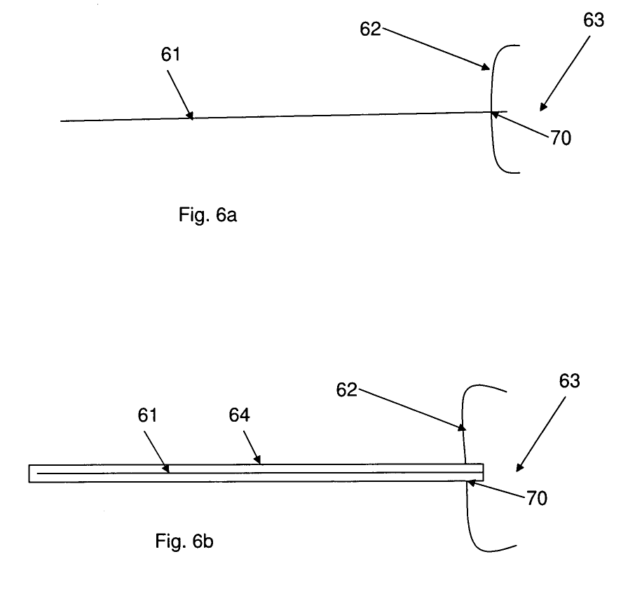

For example, FIG. 5a depicts a cannulated delivery device 50 with a retractable hook 51. The hook 54 may be retracted to position 51a until the cannulated delivery device is near the capsule wall. The hook 54 may be activated to position 51b using a pull wire 52 to attach the cannulated device 50 to the capsule wall 53.

Alternatively, referring to FIG. 5b, a cannulated delivery device 55 may be attached to the capsule wall 56 by creating a low pressure region between the capsule wall and at least a portion of the cannulated delivery device. Referring to FIG. 5c, the cannulated delivery device 55 may include one or more suctioning channels 57 along the walls of the device and a recessed space 58 at the distal end between the inner wall 55b and the outer wall 55a of the cannulated device. Although shown as a continuous ring, the recessed space may comprise one or more discrete sections. The recessed space as a whole or discrete sections are in fluid communication with one or more suctioning channels. Referring to FIG. 5b, a low pressure compartment 59 is formed by a portion of the capsule wall 60 in contact with the delivery device and the recessed space 58. Low pressure region between the capsule wall and at least a portion of the cannulated delivery device may be formed by removing air out of the low pressure compartment 59 through one or more suctioning channels 57.

In another aspect, methods of using cannulated devices described above for delivering pharmaceutical depots inside joint space are provided. In order to place the cannulated delivery device at a surgical site, a small incision may be made through the skin. A guidewire may then be inserted through the skin and is advanced to the outer wall of the joint capsule. In some embodiments, the guidewire may be gently pushed into the capsule wall or completely through the capsule. In other embodiments, the guidewire may be attached to the outer wall of the joint capsule. The cannulated delivery device can then be advanced over the guidewire to the outer wall of the joint capsule or through it. Alternatively, the cannulated device may be advanced to the outer wall of the joint capsule without the aid of the guidewire. The positioning of the guidewire, the cannulated delivery device, or both may be inserted under radiographic or image guided control to verify proper positioning at the joint capsule.

Next, the joint capsule wall is penetrated or is enlarged, if the guidewire or the delivery device have been already inserted into or through the capsule wall, to provide access for the delivery device into the joint space. The capsule wall may be cut either by the cannulated device itself or using a separate instrument. In embodiments where the cannulated device has a sharp distal end, as shown in FIGS. 4a and 4b, the cannulated device itself may be used to cut the capsule wall. In other embodiments, the capsule may be cut with a separate cutting instrument such as a steinman pin, trocar, circular punch, a scalpel, etc.

It may be advisable to pull the capsule away from the underlying joint tissue before penetrating the capsule wall. It may be achieved using the embodiment of the instant cannulated delivery device with docking members or using another device capable of being securely attached to the capsule wall. Pulling the capsule away from the joint while penetrating the capsule may prevent damage to underlying tissue. This technique also allows a practioner to make a clean and well-defined cut. Once the cut is made, the cannulated delivery device may then be advanced through the cut in the capsule wall into the joint space.

In certain embodiments, the cannulated device may be left docked to the capsule wall. If the cannulated delivery device remains docked throughout the procedure, it may or may not have the soft tip. A depot may be delivered through the docked device. Alternatively, a second cannulated device having a soft tip may be passed through the first device into the joint space.

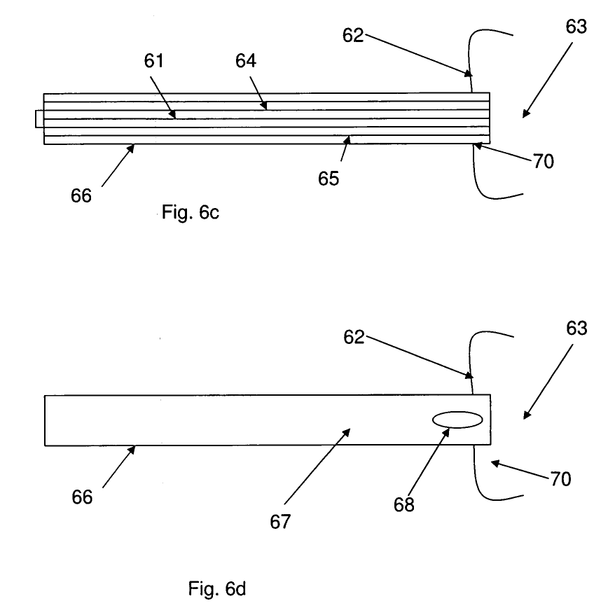

Alternatively, the capsule wall tissue may be dilated without actually cutting the tissue. Referring to FIGS. 6a to 6e, progressively larger tubes may be slid over each other until the capsule wall is open to the desired range, at which time one or all inner tubes may be removed leaving the remaining tube or tubes as a working channel for insertion of the depot. An embodiment of the cannulated delivery device with nesting tubes, as shown in FIG. 1c, may be utilized to dilate capsule wall in steps. Alternatively, individual. i.e., not connected, cannulas of progressively larger outer diameter may be used to dilate the capsule wall and to create a working channel through which the instant cannulated delivery devices or other instruments or devices may access the joint space. In some embodiments, it may be preferable to mechanically pull the capsule away from the joint while dilating an opening in the capsule wall to prevent damage to underlying tissue.

Referring to FIG. 6a, a guidewire 61 is first pushed through the capsule wall 62 into the joint space 63. This step creates an initial opening 70 in the capsule wall 66. Referring to FIG. 6b, a tube 64 is slid over the guidewire 61 to dilate the opening 70 in the capsule wall 62 into the joint space 63. Additional tubes 65 may be slid over the first tube 64 and a tube 66 may be slid over the tube 65, as necessary, to dilated the opening 70 in the capsule wall 62 into the joint space 63, as shown in FIG. 6c. Once the opening 70 in the capsule wall is dilated to the desired size, the inner tube 64, the inner tube 65, or both may be removed. By way of non-limiting example, FIG. 6d depicts an embodiment in which both tubes 64 and 65 are removed, and the remaining nesting tube 66 defines a working channel 67 for insertion depot 68 or other instruments (not shown) into the joint space. A person with ordinary skill in the art undoubtedly realizes that the number of nesting tubes, their diameter and the difference in the diameter between neighboring nesting tubes may vary depending on various factors, including, but not limited to, the type of the joint space to be accessed with the device, the size and condition of the patient, the size of the desired opening, surgeon preferences, and so forth, and is capable of selecting the appropriate number and size of the nesting tubes for a particular procedure.

As mentioned above, it is desirable to accommodate the anatomical structure of the joint capsule tissue when designing the shape of the nesting tubes to allows maximal tissue separation with minimal tissue damage. For example, in some embodiments, the cross-section of the nesting tubes may have a vertical oval shape to correspond to the collagen tissue planes in the joint capsule.

The cannulated delivery device may be used to deliver a pharmaceutical depot into the joint space. Any type of depot known and used in the art may be used. In general, the depot may comprise a depot portion that holds the therapeutic agent, an anchoring portion that extends from the depot portion to prevent migration of the depot from the target site. Examples, of suitable depots are disclosed, for example, in U.S. patent application Ser. Nos. 11/403,733 and 11/734,618. In preferred embodiments, the depot may be used to deliver an anti-inflammatory agent to the joint space.

In some embodiments, the hole or the dilated opening in the capsule wall may be closed by mechanical means. In some embodiments, the depot may be used to close the hole or the dilated opening. The depot may be positioned as to have a depot portion lodged against an inner the inner wall of the capsule and the retaining portion lodged in the hole. Alternatively, the depot may comprise a depot portion, a connecting portion, and a retaining portion, wherein the depot portion is disposed against the inner wall of the capsule, the connecting portion traverses the wall of the joint capsule, and the retaining wall is disposed against the outer wall of the joint capsule.

In other embodiments, a plug made from natural or synthetic materials may be used to close the joint capsule. The plug may be inserted into the joint through the cannulated delivery device, while the device is still in place within the joint capsule, and then pulled into the hole after the cannula is removed. The plug may be held in place by friction or may be sutured in place. Alternatively, the plug may harden in place or be adherent to the surrounding tissue.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A medical device comprising:
a cannula defining a working channel between a distal end and a proximal end, wherein the distal end comprises an atraumatic tip.

2. The device of claim 1 further comprising a docking member proximally adjacent to the distal end of the cannula.

3. The device of claim 1 wherein outside diameter of the cannula decreases toward the distal end.

4. The device of claim 1 wherein the cannula comprises a series of removable nesting tubes slidable relative to each other.

5. The device of claim 1 wherein the cross-section of the cannulated delivery device has a vertical oval shape.

6. The device of claim 1 wherein the atraumatic tip comprises a rounded tip.

7. The device of claim 1 wherein the atraumatic tip comprises a soft tip.

8. A method for delivering therapy into a joint space, the method comprises:
dilating the tissue of a capsule wall; and
delivering a depot into the joint space.

9. The method of claim 8 wherein the capsule wall is dilated with a cannulated delivery device comprising removable nesting tubes slidable relative to each other.

10. The method of claim 9 wherein one or more inner nesting tubes are removed from the delivery device to define a working channel.

11. The method of claim 8 wherein the capsule wall is dilated by sliding individual cannulas with progressively increasing diameter through an opening in the capsule wall and wherein one or more inner tubes are removed to define a working channel.

12. The method of claim 8 further comprising:
inserting a plug through the cannulated delivery device; and
pulling the plug through a dilated opening in the wall of the joint capsule while removing the cannulated device.

13. The method of claim 12 further comprising placing a depot into a dilated opening in the wall of the joint capsule.

14. The method of claim 12 wherein the cross-section of the cannulated delivery device has a vertical oval shape.

15. A method for delivering therapy into a joint space, the method comprises:
docking a first cannulated device to an outer wall of the joint capsule;
making a cut in the capsule wall; and
introducing a depot into the joint space.

16. The method of claim 15 wherein the step of introducing the depot comprises:
advancing the first cannulated device into the joint space;
delivering the depot into the joint space through the first cannulated device.

17. The method of claim 15 wherein the step of introducing the depot comprises:
introducing a second cannulated device into the joint space through the first cannulated device;
delivering the depot into the joint space through the second delivery device.

18. The method of claim 15 wherein the depot comprises a depot portion, a connecting portion, and a retaining portion, wherein the depot portion is disposed against the inner wall of the capsule, the connecting portion traverses the wall of the joint capsule, and the retaining wall is disposed against the outer wall of the joint capsule.

19. The method of claim 17 wherein the first cannulated device, the second cannulated delivery device, or both comprise a cannula defining a working channel between a distal end and a proximal end, wherein the distal end comprises an atraumatic tip.

20. The method of claim 15 further comprising a step of pulling the capsule away from the joint before making a cut in the capsule wall.
